(19) United States
(12) Patent Application Publication
(10) Pub. No.: US 2021/0151154 A1
(43) Pub. Date: May 20, 2021

(54) METHOD FOR PERSONALIZED SOCIAL ROBOT INTERACTION

(71) Applicant: Intuition Robotics, Ltd., Ramat-Gan (IL)

(72) Inventors: Itai MENDELSOHN, Ramat Gan (IL); Shay ZWEIG, Harel (IL); Dor SKULER, Ramat Gan (IL); Roy AMIR, Ramat Gan (IL)

(73) Assignee: Intuition Robotics, Ltd., Ramat-Gan (IL)

(21) Appl. No.: 17/158,802

(22) Filed: Jan. 26, 2021

Related U.S. Application Data
(63) Continuation of application No. 16/232,510, filed on Dec. 26, 2018.
(60) Provisional application No. 62/610,296, filed on Dec. 26, 2017.

(51) Int. Cl. G16H 20/00; B25J 13/08; B25J 11/00; B25J 9/16; B25J 13/00; G16H 20/30; G06Q 10/06; G06F 16/70

(52) U.S. Cl. CPC G16H 20/00; B25J 13/08; B25J 11/0005; B25J 9/1694; G06N 3/008; B25J 11/003; G16H 20/30; G06Q 10/06; G06F 16/70; B25J 13/00

(57) ABSTRACT

A system and method for personalization of an interaction between a social robot and a user. The method comprises collecting, by one or more of a plurality of sensors of the social robot, a first set of sensory data from the user; determining, based on the collected first set of sensory data, a first state of the user; determining whether the first state of the user requires a change to a second state of the user; performing, by the social robot, a first operational schema selected from a plurality of operational schemas based on an influence score of the first operational schema upon determination that the first state of the user requires a change to the second state of the user, wherein the influence score of the first operational schema is determined based on the likelihood of the first operational schema to cause a user to change from the first state to a second state; collecting, by one or more of the plurality of sensors of the social robot, a second set of sensory data from the user; and determining, based on the collected second set of sensory data, an actual state of the user.

### CROSS-REFERENCE TO RELATED APPLICATIONS

This Application is a continuation of U.S. patent application Ser. No. 16/232,510, filed Dec. 26, 2018, currently pending, which claims the benefit of U.S. Provisional Application No. 62/610,296 filed on Dec. 26, 2017, the contents of which are hereby incorporated by reference.

### TECHNICAL FIELD

The disclosure generally relates to social robots.

### BACKGROUND

Social robots are autonomous machines that interact with humans by following social behaviors and rules. The capabilities of such social robots have increased over the years and social robots are currently capable of identifying users' behavior patterns, learning users' preferences and reacting accordingly, generating electro-mechanical movements in response to user's touch, user's vocal command, and so on.

These capabilities enable social robots to be useful in many cases and scenarios such as interacting with patients that suffer from various issues, such as autism and stress problems, assisting users to initiate a variety of computer applications, and the like. Social robots often use multiple resources including microphones, speakers, display units, and the like to interact with users.

One key disadvantage of current social robots is that the influence of the robot's actions on the user's responses and behaviors is not taken into account when executing the robot's capabilities. For example, a social robot may identify that a user is bored and therefore start to play music in order to relieve the boredom. However, the robot is unable to determine the influence the music has on the state of the specific user at the specific time point. This leads, in part, to impersonalized interaction of the social robot with the user.

It would therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “certain embodiments” may be used herein to refer to a single embodiment or multiple embodiments of the disclosure.

Certain embodiments disclosed herein also include a method for personalization of an interaction between a social robot and a user. The method comprises collecting, by one or more of a plurality of sensors of the social robot, a first set of sensory data from the user; determining, based on the collected first set of sensory data, a first state of the user; determining whether the first state of the user requires a change to a second state of the user; performing, by the social robot, a first operational schema selected from a plurality of operational schemas based on an influence score of the first operational schema upon determination that the first state of the user requires a change to the second state of the user, wherein the influence score of the first operational schema is determined based on the likelihood of the first operational schema to cause a user to change from the first state to a second state; collecting, by one or more of the plurality of sensors of the social robot, a second set of sensory data from the user; and determining, based on the collected second set of sensory data, an actual state of the user.

Certain embodiments disclosed herein also include a social robot for personalization of an interaction between the social robot and a user. The social robot includes a processing circuitry; and a memory, the memory containing instructions that, when executed by the processing circuitry, configure the social robot to: collect, by one or more of a plurality of sensors of the social robot, a first set of sensory data from the user; determine, based on the collected first set of sensory data, a first state of the user; determine whether the first state of the user requires a change to a second state of the user; perform, by the social robot, a first operational schema selected from a plurality of operational schemas based on an influence score of the first operational schema upon determination that the first state of the user requires a change to the second state of the user, wherein the influence score of the first operational schema is determined based on the likelihood of the first operational schema to cause a user to change from the first state to a second state; collect, by one or more of the plurality of sensors of the social robot, a second set of sensory data from the user; and determine, based on the collected second set of sensory data, an actual state of the user.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a perspective view of a social robot for personalized interactions between a social robot and a user according to an embodiment.

FIG. 2 is a schematic block diagram of a controller embedded within a social robot and adapted to perform the disclosed embodiments.

FIG. 3 is a flowchart of a method for personalization of an interaction between a social robot and a user according to an embodiment.

FIG. 4 is a flowchart of a method for personalization of an interaction between a social robot and a user according to another embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

By way of example to some embodiments, a social robot may collect first sensory data to determine whether at least one predetermined goals to be achieved by the user has been achieved. Then, the social robot may select an operational schema having the highest priority score from a plurality of operational schemas, and perform the selected operational schema. The social robot may further select a second sensory data indicating the user's response to the performed first operational schema. The robot is further configured to determine an achievement status of the at least one goal based on the user's response and update a memory with the achievement status.

An operational schema is a plan performed by the social robot designed to cause the user to respond in a way that improves the score of the goal, i.e., that brings the user closer to achieving the predetermined goal. For example, an operational schema may include suggesting that the user to contact a family member in order to improve the user's social activity score.

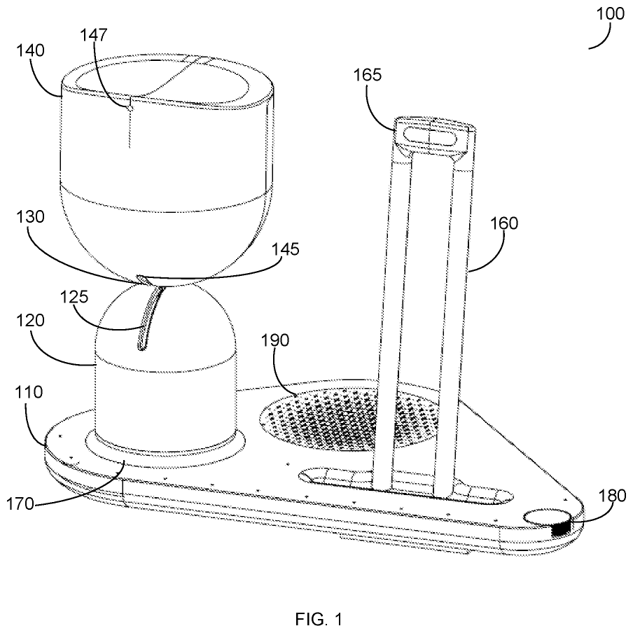

FIG. 1 is an example perspective view of a social robot 100 for performing personalization of interactions between a social robot and a user according to an embodiment.

In an example configuration, the social robot 100 includes a base 110. The base 110 is an assembly made of, for example, a rigid material, e.g. plastic, to which other components of the robot 100 are connected, mounted, or placed, as the case may be. The base 110 may include a variety of electronic components, hardware components, and the like. In example configuration, the base 110 may include a volume control knob 180, a speaker 190, and a microphone.

The social robot 100 includes a first body segment 120 mounted on the base 110 within a ring 170 designed to accept the first body segment 120. In an embodiment, the first body segment 120 is formed as a hollow hemisphere with its base configured to fit within the ring 170, though other appropriate shapes may be used. A first aperture 125, typically crossing through the apex of the hemisphere, provides access into and out of the hollow of the first body segment 120. The first body segment 120 is mounted to the base 110 within the confinement of the ring 170 such that it may rotate about its vertical axis symmetry. For example, the first body segment 120 may be able to rotate clockwise or counterclockwise relative to the base 110. The rotation of the first body segment 120 about the base 110 may be achieved by, for example, a motor (not shown) mounted to the base 110 or a motor (not shown) mounted to the first body segment 120.

The social robot 100 further includes a second body segment 140. The second body segment 140 is typically a hemisphere, although other appropriate bodies may be used, having a second aperture 145. The second aperture 145 is located at the apex of the hemisphere of the second body segment 140. When assembled, the second aperture 145 is positioned to essentially align with the first aperture 125. The second body segment 140 may be mounted to the first body segment 120 by a dynamic electro-mechanical transmission 130 protruding through and into the hollow of the second body segment 140. According to another embodiment, the second body segment 140 may be mounted to the first body segment 120 by a spring system (not shown) that may include a plurality of springs and axes associated thereto. A first camera assembly 147 may be embedded within the second body segment 140. The camera assembly 147 comprises at least one image capturing sensor.

The spring system enables a motion of the second body segment 140 with respect of the first body segment 120 in a motion that imitates at least an emotional gesture understood by the user. The combined motion of the second body segment 140 with respect of the first body segment 120 corresponds to one or more of a plurality of predetermined emotional gestures capable of being presented by such movement. The second body segment 140 is mounted to the first body segment 120 through the spring system. The combination of motions made available by the first body segment 120, the spring system and the second body segment 140, is designed to provide the perception of an emotional gesture as comprehended by the user of the apparatus 100.

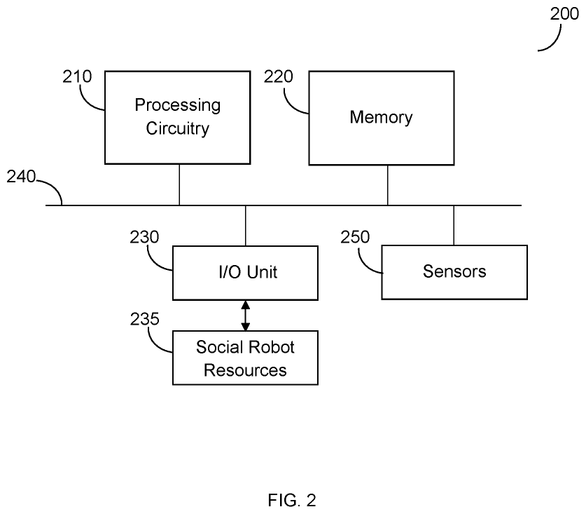

In an embodiment, a controller, not shown but further discussed below in FIG. 2, may be disposed within the first body segment 120, the second body segment 140, or the base 110 of the social robot 100.

In an embodiment, the base 110 is further equipped with a stand 160 that is designed to provide support to a portable computing device. The stand 160 may be comprised of two vertical support pillars that may include therein electronic elements, e.g. wires, sensors, and so on. A second camera assembly 165 may be embedded within a top side of the stand 160. The camera assembly 165 includes at least one image capturing sensor.

The social robot 100 may further include an audio system that includes at least a speaker 190 embedded within, for example, the base 110. The audio system may be utilized, for example, to play music, make alert sounds, play voice messages, and the like. The social robot 100 may further include an illumination system (not shown) including, one or more light emitting diodes (LEDs). The illumination system may be configured to enable the social robot 100 to support emotional gestures.

FIG. 2 is an example schematic block diagram of a controller 200 of the social robot 100 for personalization of an interaction between a social robot and a user according to an embodiment. The controller 200 includes a processing circuitry 210 that may be configured to receive sensory data, analyze the sensory data, generate outputs, etc. as further described herein below. The controller 200 further includes a memory 220. The memory 220 may contain therein instructions that when executed by the processing circuitry 210 cause the controller 200 to execute actions as further described herein below.

The processing circuitry 210 may be realized as one or more hardware logic components and circuits. For example, and without limitation, illustrative types of hardware logic components that can be used include field programmable gate arrays (FPGAs), application-specific integrated circuits (ASICs), application-specific standard products (ASSPs), system-on-a-chip systems (SOCs), general-purpose microprocessors, microcontrollers, digital signal processors (DSPs), and the like, or any other hardware logic components that can perform calculations or other manipulations of information.

In another embodiment, the memory 220 is configured to store software. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the one or more processors, cause the processing circuitry 210 to perform the various processes described herein.

The controller 200 further comprises an input/output (I/O) unit 230. The I/O unit 230 may be utilized to control one or more of the plurality of the social robot resources 235, connected thereto. The social robot resources 235 are means by which the social robot 100 collects data related to the user, interacts with the user, plays music, performs electro-mechanical movements, etc. For example, the social robot resources 235 may include sensors, electro mechanical elements, a display unit, a speaker, microphones, and the like.

In an embodiment, the I/O unit 230 may be configured to receive one or more signals captured by, e.g. sensors of the social robot 100 and send them to the processing circuitry 210 for analysis. According to one embodiment, the I/O unit 230 may be configured to analyze the signals captured by the sensors and detectors. According to yet further embodiment, the I/O unit 230 may be configured to send one or more commands to one or more of the social robot resources 235 for performing one or more capabilities of the social robot 100. The components of the controller 200 may be communicatively connected via a bus 240.

According to an embodiment, the controller 200 may be configured to collect, by one or more of a plurality of sensors 250 of the social robot 100, a first set of sensory data from the user. The sensors 250 may be for example, a microphone, a camera, a motion detector, a proximity sensor, a touch detector, and the like. The first sensory data may be one or more signals associated with the user's behavior, movements, voice, and so on. For example, the first sensory data may indicate that the user has been watching television for 12 hours during daytime, that the user has been the only person in the apartment for more than 3 days, and so on. According to another embodiment, the first set of sensory data may further include inputs received from the user while the user speaks, answers by the user to questions asked by the social robot 100, and so on.

The controller 200 may be configured to determine, based on the first sensory data, whether at least one predetermined goal to be achieved by the user has not been yet achieved. A goal is an objective that when achieved can improve the user's physical health, mental health, cognitive activity, social relationships, family bonds, and so on. For example, one of the goals may be related to physical health and the specific goal may be causing the user to perform five physical activities a day. The goals can be predefined based on the user's age, gender, current physical condition, current mental condition, and so on. The setting of such goals may be performed by the user, a care giver, and the like.

In an embodiment, the first sensory data may be analyzed using, for example, computer vision techniques for determining which of the plurality of predetermined goals were not yet achieved. The analysis may include comparing certain real-time video streams captured by one of the cameras of the social robot 100 to a predetermined index stored in the memory that may interpret the meaning of the real-time video. For example, in case the user has been the only person in the house for more than two days it may indicate that the social goal was not yet achieved.

According to one embodiment, the predetermined goals may have scores allowing to determine whether a goal was achieved or not and what is the achievement status of each goal. For example, each goal may have a score from zero to five when zero is the lowest value and five is the highest value. Five means that the goal was achieved and a value of zero to four means that the user still needs to accomplish certain activities, missions, and so on, in order to achieve a certain goal. For example, the first sensory data may indicate that two goals that still need to be achieved by the user relate to performing physical activity and maintaining social relationships. The goals may be predetermined, but they also may be changed across time with respect to the user's response to operational schemas performed by the social robot 100, associated with a certain goal, as further described herein below.

According to yet further embodiment, the controller 200 may use additional inputs other than the first sensory data for determining the score of each predetermined goal. The inputs may be, for example, information gathered online such as the weather, news and events, as well as the time of day, the user's calendar, the user's inbox, and so on. As an example, the controller 200 may identify that the physical activity goal was not yet achieved. However, an input from the user's calendar indicates that the user is about to meet a friend within 15 minutes so it may not be an appropriate time to suggest working out.

Then, the controller 200 may select a first operational schema from a plurality of predefined operational schemas. The first operational schema is determined to have a priority score that is higher than the priority scores of the rest of the plurality of operational schemas. According to one embodiment, the selected first operational schema is associated with the at least one of a plurality of predetermined goals that was not yet achieved. The operational schemas are plans performed by the social robot 100 designed to cause the user to respond in a way that improves the score of the goal, i.e., get closer to achieving the predetermined goal. For example, an operational schema may be associated with achieving a social activity goal, thus the operational schema may include suggesting the user to contact a certain friend that the user usually likes to talk with. According to the same example, the operational schema may also initiate a phone call connecting the user and the user's friend, upon the user's approval. In an embodiment, when two or more operational schemas share the same priority score, the controller 200 may select the operational schema to be performed randomly.

The priority score of each operational schema may be determined based on a set of rules, user's preferences, historical data associated with the user, historical data associated with a plurality of users having similar properties such as the user, a combination thereof, and so on. The set of rules may determine that, for example, when the social activity goal is incomplete, but it is currently nighttime, the score of the operational schema that suggests calling a friend may be relatively low comparing to an operational schema that suggests to login to a social network website, such as Facebook®.

The user's preferences may be learned by the social robot 100 and may include using historical data to identify the user's preferences. For example, historical data may indicate that during the morning hours the user responds in a positive manner to operational schemas that suggest listening to music, which improves the score of an entertainment goal. Therefore, during morning hours the score of an operational schema that suggests listening to music may be relatively high comparing to an operational schema suggesting, for example, to read a book.

The historical data gathered from a plurality of social robots associated with a plurality of different users, having similar properties such as the user, may allow determining the priority score of at least a portion of the optional operational schemas of the social robot 100. The plurality of users may be other people apart from the user of the social robot 100 that already used their social robots and therefore users' preferences were identified and priority scores for each of the operational schemas were determined. The similar properties may include the users' age, gender, physical condition, mental condition, and so on. For example, the historical data gathered from the plurality of users may indicate that people from a certain state, at a certain age, enjoy listening to jazz music in the evening. Therefore, the operational schema of playing jazz music in the evening, for the user having similar properties such as the plurality of users, may receive a relatively high priority score.

According to another embodiment, the priority score of each operational schema may be determined by the controller 200 using machine-learning techniques, such as a deep learning technique. The machine learning technique may calculate multiple parameters such as the weather, time of day, user's historical health issues, other execution and scores of other operational schemas, and the like, in order to determine the priority score for each operational schema.

Then, the controller 200 may be configured to perform by the social robot 100 a first operational schema selected from a plurality of operational schemas. The controller 200 may cause at least one of the social robot resources 235 to perform the first operational schema. For example, in order to perform an operational schema that suggests the user to call a friend, the controller 200 may use the speaker, the microphone, and so on.

The controller 200 may be further configured to collect by one or more of the plurality of sensors of the social robot 100 a second sensory data from the user. The second sensory data may be one or more signals associated with the user's behavior, movements, voice, etc. that indicate the user's response to the first operational schema. For example, after a first operational schema has suggested performing a physical activity, the collected second sensory data indicates that the user had completed the entire physical activity.

The controller 200 is further configured to determine an achievement status of each of the predetermined goals based on the user's response. The achievement status may be a score indicative of the gap between the current state and full achievement of the predetermined goal. For example, the current state of a physical activity goal may be incomplete as only four out of five required physical activities were completed. However, after the fifth physical activity is completed the achievement status may indicate that the physical activity goal was achieved. Thereafter, the controller 200 may update the memory 220 with the achievement status.

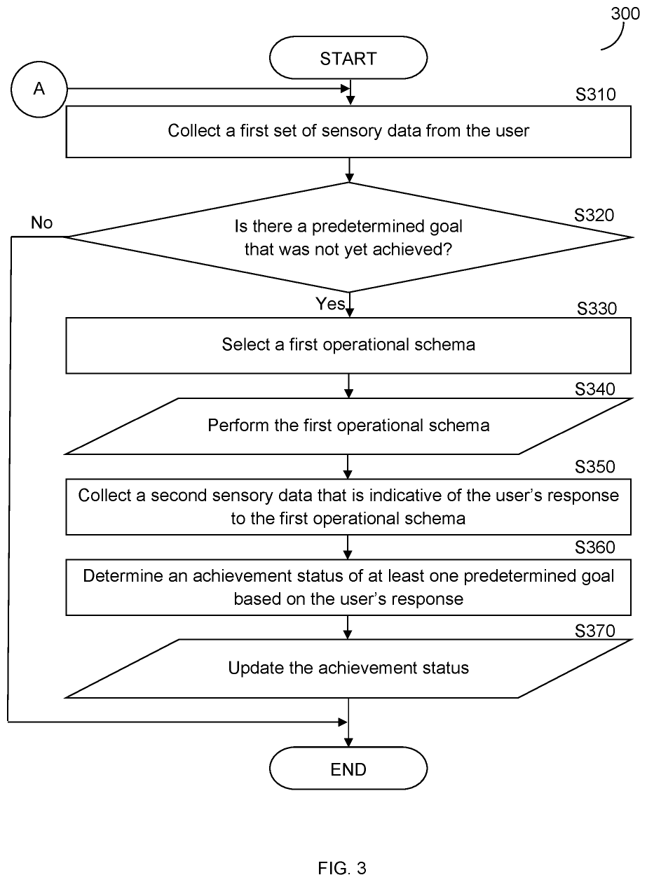

FIG. 3 shows an example flowchart 300 of a method for personalizing interactions between a social robot and a user according to an embodiment. At S310, a first set of sensory data related to a user is collected. The collection is performed using the plurality of sensors of the social robot 100 as further described herein above with respect of FIG. 2.

At S320, it is determined based on the first sensory data whether at least one predetermined goal to be achieved by the user has not yet been achieved, and if so execution continues with S330; otherwise, execution continues with S380.

At S330, a first operational schema is selected from a plurality of operational schemas. An operational schema includes commands to be performed by a social robot designed to cause the user to respond in a way that improves the score of a goal, i.e., bringing the user closer to achieving the predetermined goal. For example, an operational schema may include suggesting the user to contact a family member in order to improve the user's social activity score.

The first operational schema is determined to have a priority score that is higher than the priority scores of the rest of the plurality of operational schemas. In an embodiment, when two or more operational schemas share the same priority score an operational schema may be chosen to be performed randomly from among the two or more operational schemas. At S340, the first operational schema is performed by the social robot 100.

At S350, a second sensory data indicating the user's response to the first operational schema is collected by one or more of the plurality of sensors of the social robot. At S360, an achievement status of at least one of the plurality of predetermined goals is determined based on the user's response. At S370, the achievement status is updated, e.g., within a memory.

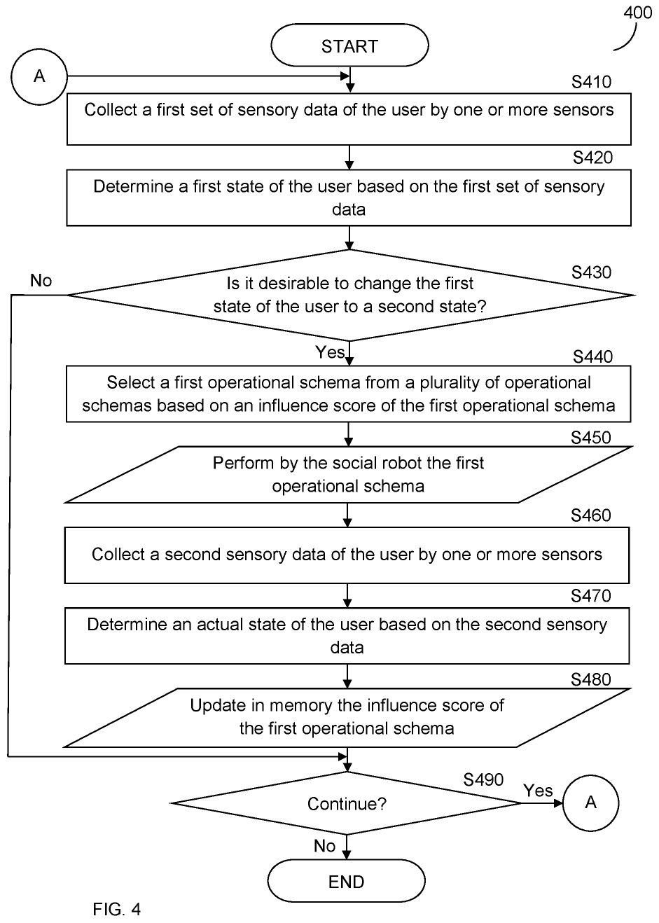

FIG. 4 shows an example flowchart 400 of a method for personalized adaptation of an interaction with a user and a social robot according to an embodiment. At S410, a first set of sensory data of the user is collected, e.g., by one or more of a plurality of sensors of a social robot as further described herein above with respect of FIG. 2.

At S420, a first state of the user is determined based on the collected first sensory data. In an embodiment, S420 may further include the step of analyzing the collected first set of sensory data. The first state of the user represents at least one of a mood of the user, a certain behavior of the user, a certain behavior pattern, a user's feeling, and the like. For example, the user state may be categorized as sadness, happiness, boredom, loneliness, etc. As an example, the first set of sensory data indicates that the user has been sitting on a couch for 5 hours, that the user has been watching the TV for more than 4.5 hours, and that the current time is 1:00 pm, the user's first state is able to be determined.

At S430, it is checked whether the first state of the user requires a change to a second state of the user and if so, execution continues with S440; otherwise, execution continues with S490. The second state of the user is a better state. That is, if the first user state indicates that the user has been sitting on the couch for 5 hours, the second user state may be achieving a goal of performing at least three physical activities a day.

The determination of whether the first user state requires a change to a second user state or not may be achieved using a predetermined threshold. That is, if certain predetermined parameters of the first user state were identified within the first set of sensory data, it is determined that the threshold was crossed, and therefore a change is required. For example, the predetermined threshold may determine that in case two parameters that indicate a loneliness state are identified within the first set of sensory data, the threshold is crossed and therefore a change is required. According to the same example, the loneliness parameters may be, for example, a situation at which the user has been the only person in the house for more than 24 hours, the user has not been talking on the phone for more than 12 hours during the day, and so on.

At S440, a first operational schema is selected from a plurality of operational schemas based on an influence score of the first operational schema. The operational schemas are plans performed by a social robot designed to cause the user to respond in a way that improves the state of the user, i.e., change it from first state to a second state, as further described herein below. The social robot may be configured to perform at least one of a motion, play a video, etc. for executing the first operational schema. The influence score is an indication of the likelihood of the first operational schema to cause a user to change from the first user state to the second user state. For example, if it is determined that the user is not active enough, it is suggested to the user to go for a walk in order to improve the user's current state. According to the same example, the operational schema of suggesting going for a walk may be chosen from a plurality of operational schemas stored in the memory 220, based on the influence score related thereto. The influence score may be determined based on past experience, learned user's patterns, and so on.

At S450, the first operational schema, selected from a plurality of operational schemas based on the influence score of the operational schema, is performed, e.g., by the social robot. At least one of the social robot resources may be used to perform the first operational schema. For example, in order to perform an operational schema that suggests the user call a friend, the controller of the social robot may use the speaker, the microphone, and so on.

At S460, a second sensory data of the user is collected, e.g., by at least one of a plurality of sensors of the social robot. The second sensory data may be indicative of the response of the user to the first operational schema. The second sensory data is indicative of the user's response to the execution of the first operational schema. For example, the second sensory data may indicate that the user called a specific friend after the first operational schema reminded the user to maintain a relationship with the specific friend.

At S470, an actual state of the user is determined based on the second sensory data. The actual state of the user represents the realistic feeling, mood, behavior, and so on of the user. The state may be determined in real time, while the first operational schema is executed, right after the execution ends, and so on. That is to say, the actual state may indicate the response, or the reaction, of the user responsive to the execution of the first operational schema.

In an embodiment, determination of the user's actual state may be achieved by comparing the collected second sensory data to a plurality of users' reactions that were previously analyzed, classified and stored in a database. The previously analyzed users' reactions may include, e.g., visual parameters that are indicative of sadness state, happiness state, active state, etc.

As an example, it may be determined that the first user state indicates that the user is sad, and therefore a suitable operational schema, such as playing music, is selected based on an influence score related thereto. According to the same example, after the second sensory data is collected and the actual user state is determined, an actual improvement with the user's state is identified. That is to say, the influence level of the operational schemas is determined, as executed by the social robot 100, on the user's state, i.e., their feeling, behavior, mood, etc.

At S480, the influence score of the first operational schema is updated, e.g., in the memory of the social robot, based on the schema's ability to cause the user to reach the second state from the first state and further based on the actual state. At S490, it checked whether to continue the operation and if so, execution continues with S410; otherwise, execution terminates.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; A and B in combination; B and C in combination; A and C in combination; or A, B, and C in combination.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

What is claimed is:

1. A method for personalization of an interaction between a social robot and a user, comprising:
collecting, by one or more of a plurality of sensors of the social robot, a first set of sensory data from the user;
determining, based on the collected first set of sensory data, a first state of the user;
determining whether the first state of the user requires a change to a second state of the user;
performing, by the social robot, a first operational schema selected from a plurality of operational schemas based on an influence score of the first operational schema upon determination that the first state of the user requires a change to the second state of the user, wherein the influence score of the first operational schema is determined based on the likelihood of the first operational schema to cause a user to change from the first state to a second state;
collecting, by one or more of the plurality of sensors of the social robot, a second set of sensory data from the user; and
determining, based on the collected second set of sensory data, an actual state of the user.

2. The method of claim 1, further comprising:
updating in a memory the influence score of the first operational schema based on a response of a user to reach the second state from the first state.

3. The method of claim 1, wherein the influence score is further updated based on the actual state.

4. The method of claim 1, wherein determining whether the first state of the user requires a change to the second state of the user further comprises:
identifying at least one predetermined parameter of the first user state in the first set of sensory data; and
determining that the first state of the user requires a change to the second state of the user when a predetermined threshold is crossed.

5. The method of claim 1, wherein determining the actual state of the user further comprises:
comparing the collected second sensory data to a plurality of reactions of users that were previously analyzed, classified and stored in a database.

6. A non-transitory computer readable medium having stored thereon instructions for causing a processing circuitry to perform a process for personalization of an interaction between a social robot and a user, the process comprising:
collecting, by one or more of a plurality of sensors of the social robot, a first set of sensory data from the user;
determining, based on the collected first set of sensory data, a first state of the user;
determining whether the first state of the user requires a change to a second state of the user;
performing, by the social robot, a first operational schema selected from a plurality of operational schemas based on an influence score of the first operational schema upon determination that the first state of the user requires a change to the second state of the user, wherein the influence score of the first operational schema is determined based on the likelihood of the first operational schema to cause a user to change from the first state to a second state;
collecting, by one or more of the plurality of sensors of the social robot, a second set of sensory data from the user; and
determining, based on the collected second set of sensory data, an actual state of the user.

7. A social robot for personalization of an interaction between the social robot and a user, comprising:
a processing circuitry; and
a memory, the memory containing instructions that, when executed by the processing circuitry, configure the social robot to:
collect, by one or more of a plurality of sensors of the social robot, a first set of sensory data from the user;
determine, based on the collected first set of sensory data, a first state of the user;
determine whether the first state of the user requires a change to a second state of the user;
perform, by the social robot, a first operational schema selected from a plurality of operational schemas based on an influence score of the first operational schema upon determination that the first state of the user requires a change to the second state of the user, wherein the influence score of the first operational schema is determined based on the likelihood of the first operational schema to cause a user to change from the first state to a second state;
collect, by one or more of the plurality of sensors of the social robot, a second set of sensory data from the user; and
determine, based on the collected second set of sensory data, an actual state of the user.

8. The social robot of claim 7, wherein the social robot if further configured to:
update in a memory the influence score of the first operational schema based on its ability to cause a user to reach the second state from the first state.

9. The social robot of claim 7, wherein the influence score is further updated based on the actual state.

10. The social robot of claim 7, wherein the social robot if further configured to:
identify at least one predetermined parameter of the first user state in the first set of sensory data; and,
determine that the first state of the user requires a change to the second state of the user when a predetermined threshold is crossed.

11. The social robot of claim 7, wherein the social robot if further configured to:
compare the collected second sensory data to a plurality of reactions of users that were previously analyzed, classified and stored in a database for determining the actual state of the user.
